(19) United States
(12) Patent Application Publication
(10) Pub. No.: US 2013/0023885 A1
(43) Pub. Date: Jan. 24, 2013

(54) Unidirectional Plunger Device For Syringe

(71) Applicants: Kevin Madden, Limerick (IE); John Henry, Limerick (IE); Andreas Legner, Weinbach (DE)

(72) Inventors: Kevin Madden, Limerick (IE); John Henry, Limerick (IE); Andreas Legner, Weinbach (DE)

(73) Assignee: HOWMEDICA OSTEONICS CORP., Mahwah, NJ (US)

(21) Appl. No.: 13/187,978

(22) Filed: Jul. 21, 2011

(51) Int. Cl. A61B 17/58; B23P 17/04

(52) U.S. Cl. 606/93; 29/428

(57) ABSTRACT

A unidirectional plunger device for a syringe includes a base member and a ratchet mechanism. The base member is configured to attach to the proximal end of the syringe body such that the ratchet mechanism engages the plunger of the syringe. The ratchet mechanism may include a pawl configured to engage teeth on a plunger rod of the plunger. The ratchet mechanism is desirably configured to permit distal movement of the plunger while resisting proximal movement of the plunger. The unidirectional plunger device may be a retrofit that can be attached to (e.g., clipped onto) an existing syringe design. The device may be used in conjunction with a two-barrel syringe for dispensing a two-part curable composition, such as calcium phosphate bone cement or epoxy resin.

### BACKGROUND OF THE INVENTION

The present invention relates to syringes and to apparatus for resisting proximal movement of plungers of such syringes.

Conventionally, syringes, such as syringes for dispensing bone cement, may be provided to a user in a prefilled state. For example, syringes for dispensing two-part curable bone cement compositions may have two barrels for separately containing components of those compositions that will cure upon being combined. An example of such a syringe is disclosed in U.S. Provisional Patent Application No. 61/378,451 filed Aug. 31, 2010 (hereinafter “the '451 application”), the disclosure of which is hereby incorporated herein by reference. The body of such a multi-barrel syringe may be provided to the user with the components of the bone cement already located in the syringe barrels.

Before providing a prefilled syringe to the user, the syringe may undergo several processing steps. For example, a sealing system like that disclosed in the '451 application may be attached to the distal end of the syringe body, and the prefilled body may be sterilized (e.g., by exposure to gamma radiation). The syringe body and other components of the syringe system may also be secured into a specially designed tray or package, which integrated package is shipped to the user.

In order to dispense the bone cement composition from the syringe system, the user may first need to assemble portions of the system. For example, as disclosed in the '451 application, the user may detach the sealing system from the distal end of the syringe body and attach a static mixer in its place. The user may then advance the plunger of the syringe, which causes the component fluids of the cement composition to pass through the static mixer, in which the components are mixed together, and then be ejected from the distal end of the system.

Although effort has been devoted in the art heretofore to optimization of such syringe systems and their packaging, still further improvement would be desirable.

### BRIEF SUMMARY OF THE INVENTION

One aspect of the present invention provides an apparatus for resisting proximal movement of a plunger of a syringe, which syringe includes a body having a proximal end and a distal end. The apparatus according to this aspect of the invention desirably includes a base member and a ratchet mechanism connected to the base member. The base member is preferably adapted to securely attach to an exterior surface of the body of the syringe. The ratchet mechanism is preferably structured and arranged to contact the plunger so as to permit distal movement of the plunger with respect to the body but resist proximal movement of the plunger with respect to the body.

According to one aspect of the invention, the base member is shaped to securely attach to the proximal end of the body. In accordance with this aspect of the invention, a shape of the base member along a plane perpendicular to a longitudinal axis of the syringe body substantially matches a shape of the syringe body along that plane.

According to another aspect of the invention, the base member includes a passage therethrough, which passage is adapted to receive a portion of the plunger therethrough, and which passage is aligned with an opening at the proximal end of the syringe through which a portion of the plunger is received. In accordance with this aspect of the invention, the passage through the base member preferably has substantially the same geometry as the opening at the proximal end of the syringe body. In another alternative, the proximal end of the syringe body may have multiple openings, the plunger may have an associated plurality of plunger rods, and the base member may include a plurality of passages therethrough, the passages being aligned with the respective openings and adapted to receive the respective plunger rods therethrough.

According to yet another aspect of the invention, the apparatus may further comprise a clip connected to the base member. The clip, in accordance with this aspect of the invention, is desirably structured and arranged to securely attach to a feature on the exterior surface of the syringe body. According to this aspect of the invention, that feature may include a portion of a gripping member extending away from a longitudinal axis of the syringe body.

In accordance with yet a further aspect of the invention, the ratchet mechanism includes a pawl adapted to engage a plunger rod of the plunger. According to this aspect of the invention, the pawl is adapted to engage a plurality of teeth on the plunger rod.

According to yet further aspects of the invention, the base member may include at least one projection adapted to engage a plunger rod of the plunger. In accordance with this aspect of the invention, the plunger stabilizes an orientation of the plunger rod with respect to the syringe body. Also in accordance with this aspect of the invention, the projection may extend into the passage of the base member.

In accordance with additional aspects of the invention, the base member is detachable from the exterior surface of the syringe body.

Further aspects of the invention provide a syringe. A syringe in accordance with such an aspect of the invention includes a body, a plunger, and an apparatus in accordance with any of the above aspects of the invention. The body of such a syringe preferably has a proximal end, a distal end, and an exterior surface. The body also desirably includes at least one barrel, each of which defines a fluid channel. The plunger of the syringe according to this aspect of the invention preferably has at least one plunger rod being slidably receivable at least partially within a respective fluid channel. The base member of the apparatus in accordance with this aspect of the invention desirably is securely attached to the exterior surface of the body.

Yet further aspects of the invention provide a method for modifying a syringe. The method according to this aspect of the invention desirably includes securely attaching the base member of an apparatus in accordance with any of the above aspects of the invention to an exterior surface of a body of a syringe. The base member is preferably attached to the exterior surface such that the ratchet mechanism of the apparatus contacts a plunger of the syringe. Desirably, the contact between the ratchet mechanism and the plunger is such that distal movement of the plunger with respect to the body is permitted, but proximal movement of the plunger with respect to the body is resisted.

### BRIEF DESCRIPTION OF THE DRAWINGS

FIG. 1 is a perspective view of a portion of a syringe system in accordance with one embodiment of the invention.

FIG. 2A is a perspective view of the syringe body of the portion of the syringe system illustrated in FIG. 1.

FIG. 2B is a side view of the syringe body of FIG. 2A.

FIG. 2C is a rear view of the syringe body of FIG. 2A.

FIG. 3 is a perspective view of a portion of a syringe system including a unidirectional plunger device in accordance with one embodiment of the invention.

FIGS. 4A and 4B are perspective views of the unidirectional plunger device of FIG. 3.

FIG. 5A is a perspective view of the syringe body and the unidirectional plunger device of the portion of the syringe system illustrated in FIG. 3.

FIG. 5B is a side view of the syringe body and unidirectional plunger device of FIG. 5A.

FIG. 5C is a rear view of the syringe body and unidirectional plunger device of FIG. 5A.

FIG. 6A is a perspective view of the plunger of the portion of the syringe system illustrated in FIG. 3.

FIG. 6B is a side view of the plunger of FIG. 6A.

FIG. 7 is a perspective view of a portion of the portion of the syringe system including the unidirectional plunger device illustrated in FIG. 3.

FIG. 8A is a perspective view of a portion of the plunger illustrated in FIG. 6A.

FIG. 8B is a perspective view of a portion of the unidirectional plunger device illustrated in FIGS. 4A-B.

### DETAILED DESCRIPTION

As used herein, the term “distal” relates to the direction away from the operator of the syringe during use, while the term “proximal” relates to the direction towards the operator.

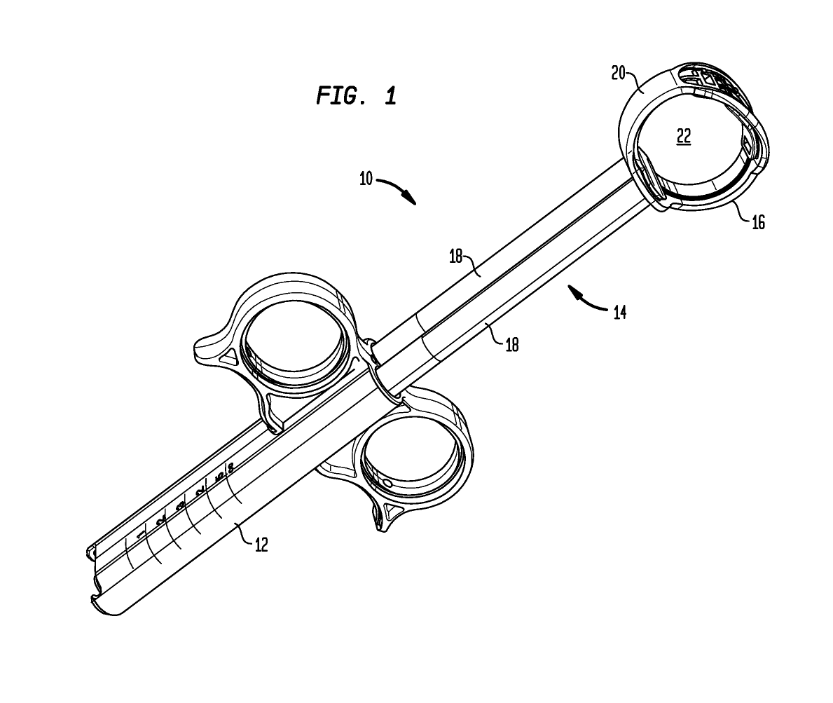

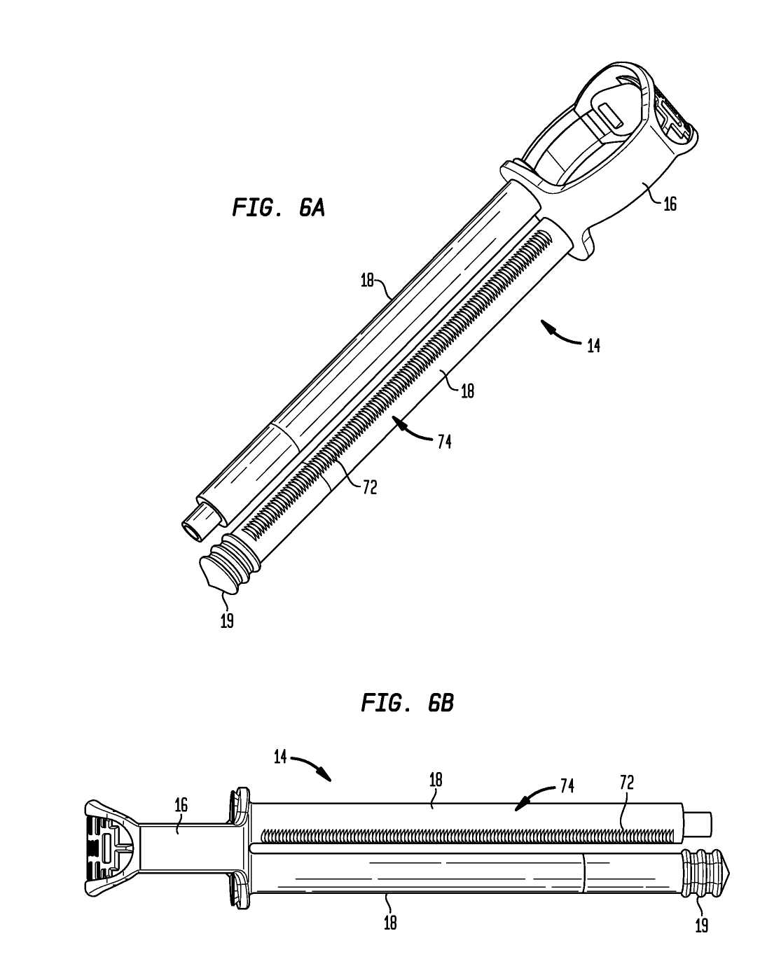

FIG. 1 illustrates a portion of a syringe system 10, such as that disclosed in the '451 application, including a syringe body 12 and a plunger 14. The plunger includes a grip element 16 and two plunger rods 18, which may all be integrally formed together (e.g., from a polymer material, such as polyamide). The plunger may also include a plunger tip 19 (see FIGS. 6A-B) located at the distal end of each plunger rod 18 and formed from an elastomeric material such as silicone rubber. The grip element 16 may include a loop 20 defining an opening 22 dimensioned to receive at least one operator's finger. Loop 20 allows an operator to grab plunger 14 and push or pull it with respect to body 12. Each of the plunger rods 18 may have a substantially cylindrical shape.

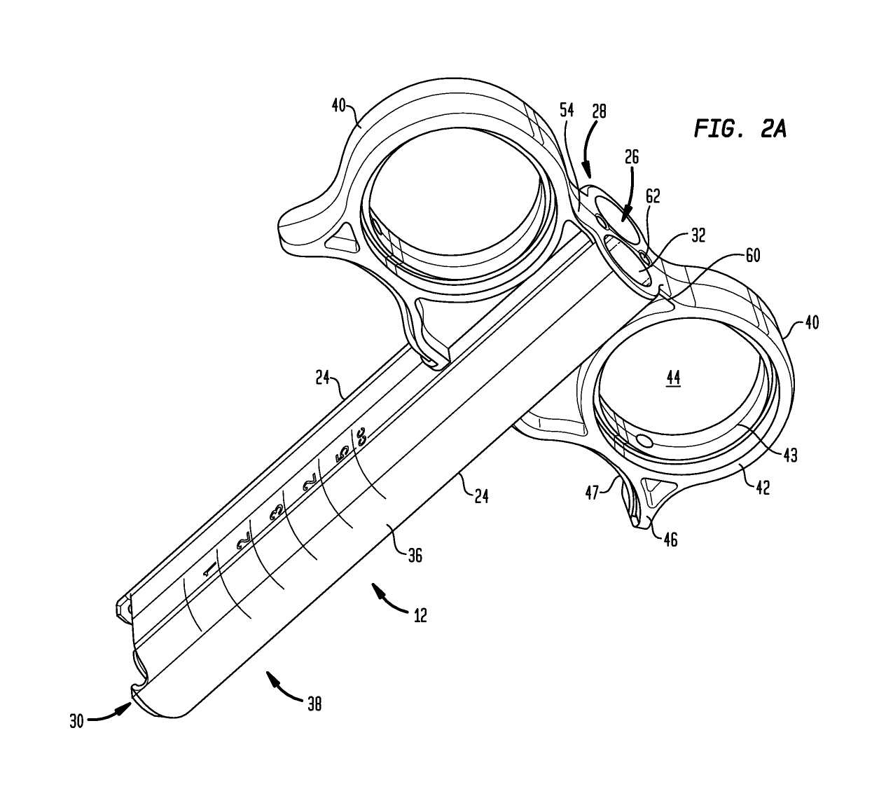

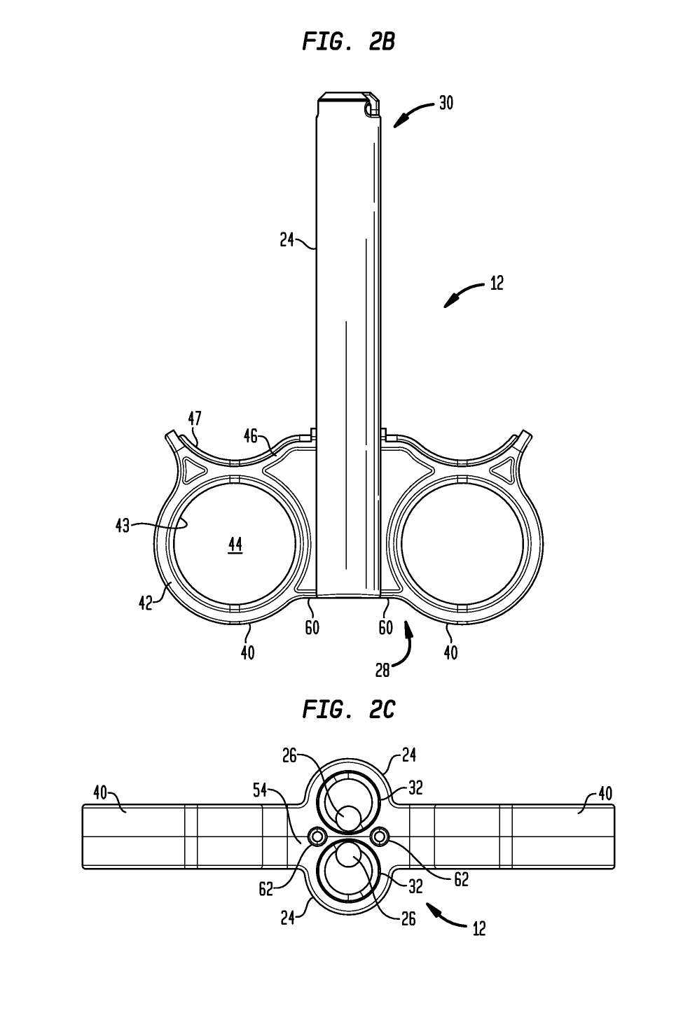

As shown in FIGS. 2A-C, the body 12 has two barrels 24, each defining a substantially cylindrical channel 26 extending through the body 12 between its proximal end 28 and its distal end 30. The channels 26 are configured to separately hold different components of a multi-part curable composition to be dispensed by the syringe system 10, such as two-part calcium phosphate cement or two-part epoxy resin. The channels 26 slidably receive the respective plunger rods 18 therein, with each of the plunger tips 19 sealingly engaing the inner periphery of the respective barrel 24, such that distal movement of the plunger 14 displaces the components of the curable composition and ejects them from the distal end 30 of the body 12. The channels 26 terminate in openings 32 at the proximal end 28 of the syringe body 12 through which the plunger rods 18 pass. A septum or internal wall 34 separates the channels 26 and prevents the separately contained fluids from mixing together.

The distal end 30 of the body 12 may be configured to connect to other components, such as a sealing system and a static mixer, as disclosed in the '451 application. An outer surface 36 of the body 12 may include markers or indicia 38 for measuring the volume of the components contained in the channels 26. The body 12 may also include gripping members 40 positioned on substantially opposite sides of the body 12 and extending radially outwardly from the outer surface 36. Each gripping member 40 may include a loop 42 defining an aperture or hole 44 and an undulating bar or element 46. The hole 44 and undulating bar 46 may each be dimensioned to receive at least one operator's finger.

The body 12 is preferably transparent and may be formed from materials such as cyclic olefin copolymer or glass. An overmolded material (not shown) may also cover all or a portion of the body 12. For example, a thermoplastic elastomer may be overmolded onto the inner surface 43 of each loop 42 and/or onto the distal surface 47 of each undulating bar 46. Such an overmolded material, which may or may not define a textured surface, may increase the grip between the operator's fingers and the syringe body 12.

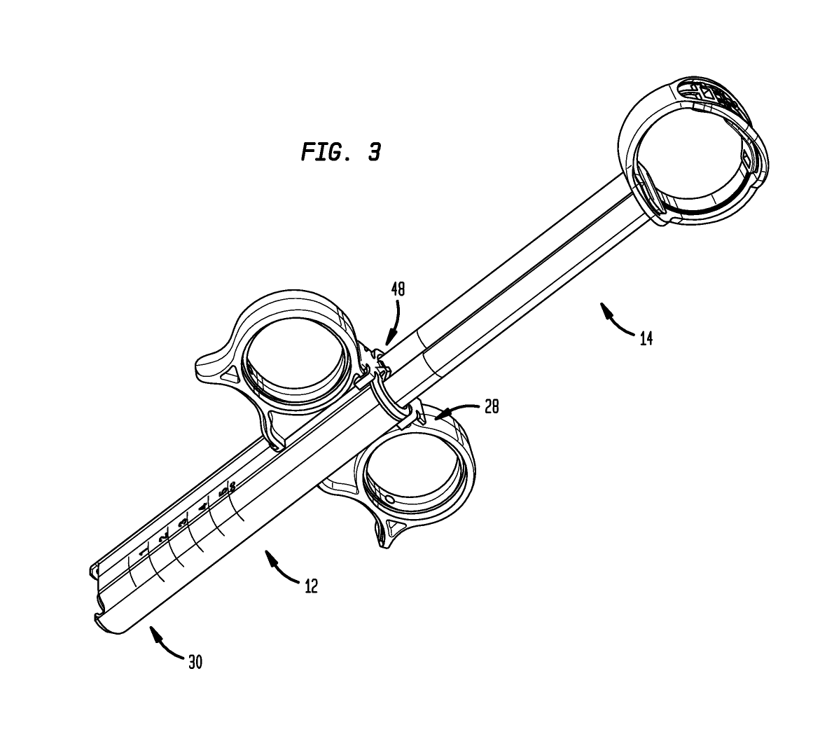

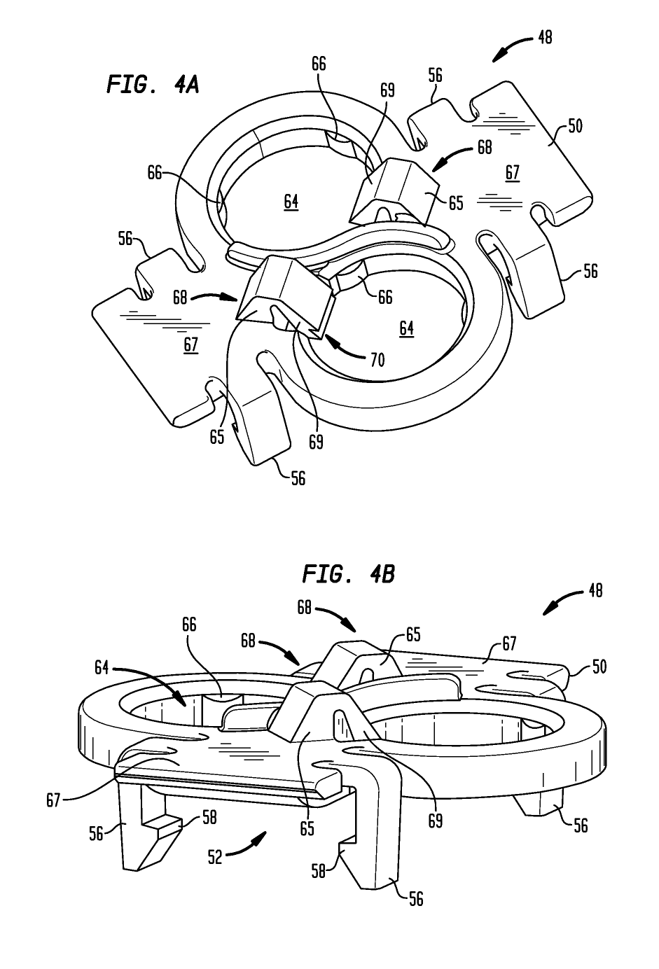

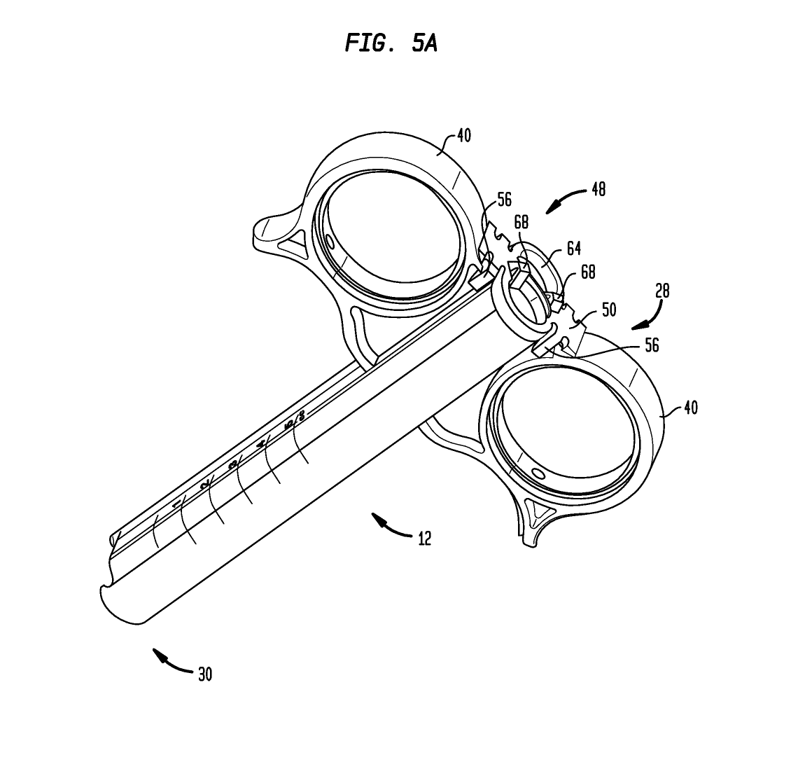

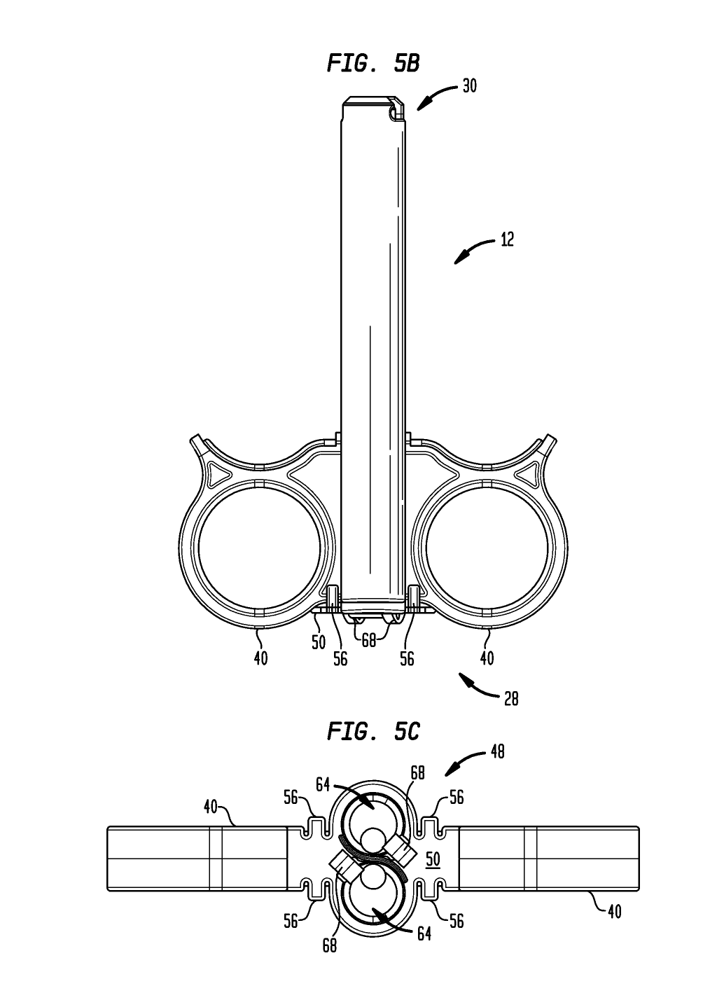

FIG. 3 illustrates the portion of the syringe system 10 illustrated in FIG. 1 in which a unidirectional plunger device 48 is attached to the proximal end 28 of the syringe body 12. Views of the unidirectional plunger device 48 isolated from the syringe body 12 are illustrated in FIGS. 4A and 4B. Various views of the unidirectional plunger device 48 attached to the proximal end 28 of the syringe body 12 are illustrated in FIGS. 5A-C.

The unidirectional plunger device 48 includes a base member 50, which may be in the form of a generally planar component that generally matches the shape of at least a portion of the syringe body 12 at the proximal end 28. The unidirectional plunger device 48 is configured to be securely attached to the proximal end 28 of the body 12, with the underside 52 of the base member 50 abutting the proximal end surface 54 of the body 12 in the region adjacent the openings 32 (see FIGS. 2A-C). The unidirectional plunger device 48 may be attached to the proximal end 28 of the body 12 by way of a clip comprising multiple (e.g., four) opposing, flexible snap latches 56 extending distally from the base member 50. Each snap latch 56 may include a bump 58 at its distal end for interlocking with a lip 60 (see FIGS. 2A-C) at the proximal end 28 of the body 12, so as to secure the unidirectional plunger device 48 to the proximal end 28. The lip 60 may be a portion of a gripping member 40.

The snap latches 56 are preferably designed so as to securely affix the unidirectional plunger device 48 to the proximal end 28 of the syringe body 12, although the snap latches 56 may also allow the unidirectional plunger device 48 to be detached from the body 12 (if desired) upon manipulation of the snap latches 56 by a user. The underside 52 of the base member 50 may also include one or more distally extending projections (not shown) configured to be received by corresponding recesses 62 (see FIGS. 2A-C) in the proximal end 28 of the body 12, in order to provide further stability for the unidirectional plunger device 48.

The unidirectional plunger device 48 may include two passages 64 through the base member 50, which passages 64 are aligned with the respective openings 32 in the proximal end 28 of the body 12, such that the plunger rods 18 pass through the passages 64. The passages 64 may each have a generally circular cross-sectional shape that substantially matches the size and shape of the openings 32. The unidirectional plunger device 48 may also include one or more (e.g., three) projections 66 spaced around each of the passages 64 in the base member 50. Each of the projections 66 is desirably configured to engage one of the plunger rods 18 so as to provide stability to that plunger rod 18, and, in turn, the entire plunger 14. That is, the combined engagement of all of the projections 66 against a particular plunger rod 18 preferably constrains the lateral position of that plunger rod 18 within the passage 64, which desirably reduces wobbling and maintains the plunger rod 18 in an axial alignment within the respective channel 26.

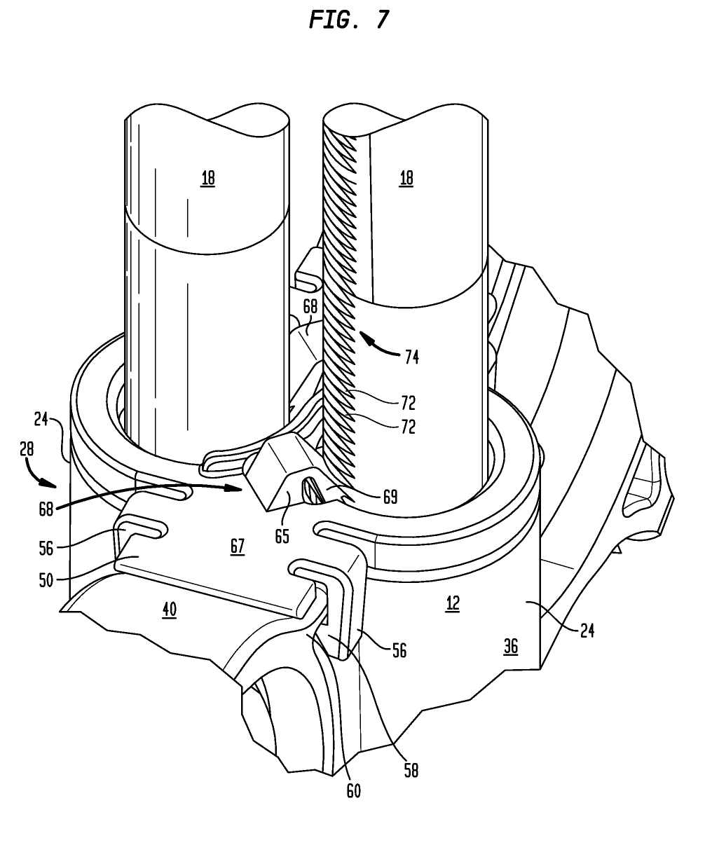

The unidirectional plunger device 48 may also include a ratchet mechanism configured to engage at least one of the plunger rods 18 when the unidirectional plunger device 48 is attached to the proximal end 28 of the syringe body 12. In one embodiment, the ratchet mechanism may include two pawls 68, each located along the periphery of a respective passage 64. Each pawl 68 includes a base 65 projecting proximally from the top surface 67 of the base member 50 and an arm 69 extending inward from the base 65 towards the center of the passage 64. The arms 69 are preferably flexible, such that the arms 69 can pivot with respect to the base 65. Each arm has a free end 70 configured to engage a respective plunger rod 18. Each arm 69 extends from the base 65 towards the respective plunger rod 18 in the distal direction and at an acute angle with respect to the longitudinal axis of the plunger rod 18. The height of the base 65 and the configuration of the arm 69 may be such that the free end 70 of the arm does not extend distally of the underside 52 of the base member 50. The free ends 70 of the arms 69 are preferably shaped to engage teeth 72 (see FIGS. 6A-B) provided along the plunger rods 18. As shown in FIGS. 6A-B, the teeth 72 may be arranged in a linear series 74 extending along all or a portion of each of the plunger rods 18. FIG. 7 illustrates the engagement between the pawls 68 and the teeth 72.

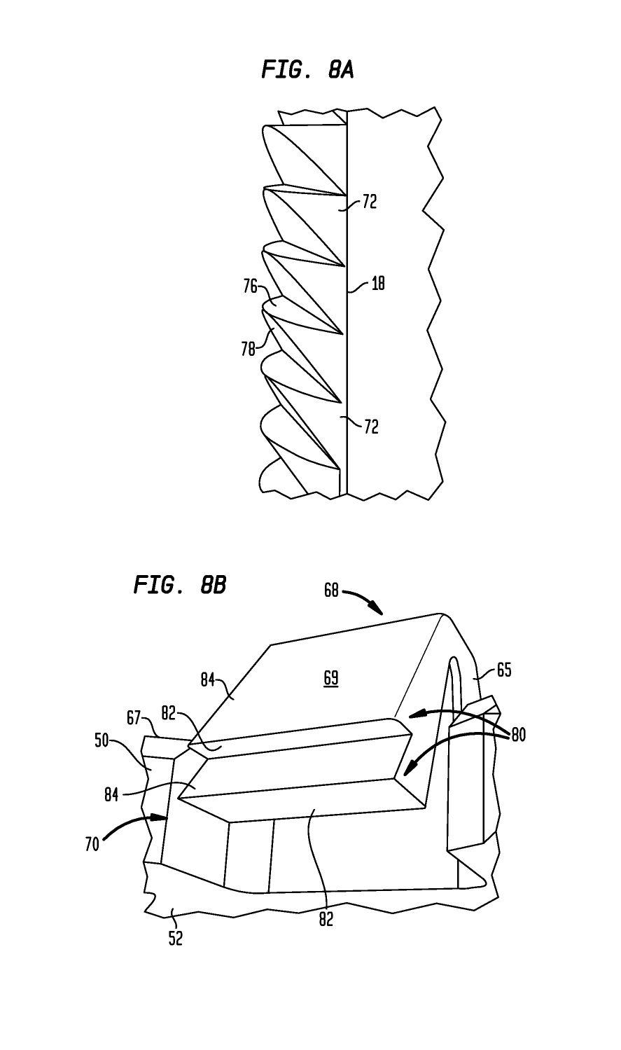

As illustrated in FIG. 8A, the teeth 72 may each include a proximally oriented face 76 and a distally oriented face 78. The proximally oriented face 76 may be oriented substantially perpendicular to the longitudinal axis of the respective plunger rod 18, and the distally oriented face 78 may define an acute angle with respect to that longitudinal axis. As illustrated in FIG. 8B, the free end 70 of each pawl 68 may be shaped so as to define one or more (e.g., two) tooth engagement portions 80. Each tooth engagement portion 80 may include a distally oriented face 82 and a proximally oriented face 84. The distally oriented face 82 may be oriented substantially perpendicular to the longitudinal axis of the respective plunger rod 18, and the proximally oriented face 84 may define an acute angle with respect to that longitudinal axis.

Preferably the engagement between the pawls 68 and the teeth 72 is such that the plunger 14 is permitted to move distally with respect to the body 12, while proximal movement of the plunger 14 is resisted. In this regard, the engagement between the distally oriented faces 82 of the tooth engagement portions 80 of the pawls 68 and the proximally oriented faces 76 of teeth 72 resists proximal movement of the plunger 14. However, when the user pushes the plunger 14 distally, the engagement between the angled distally oriented faces 78 of the teeth 72 and the angled proximally oriented faces 84 of the pawls 68 causes the arms 69 of the pawls 68 to pivot distally, such that their free ends 70 move away from the longitudinal axes of the respective plunger rods 18 and permit the plunger rods 18 to move distally.

Although the unidirectional plunger device resists proximal movement of the plunger 14, this feature may be bypassed by a user if desired. In one example, by twisting the plunger 14 illustrated in FIGS. 3 and 7 in a counterclockwise direction while pulling in the proximal direction, each series 74 of teeth 72 may be moved out of engagement with the respective pawl 68, thereby allowing the user to more easily move the plunger 14 proximally. If the material of the plunger 14 is sufficiently flexible, this bypassing may occur due to the twisting of the plunger 14 causing each of the plunger rods 18 to rotate slightly counterclockwise until the engagement between the pawls 68 and the teeth 72 is reduced or eliminated.

Some or all components of the unidirectional plunger device 48 (e.g., the base member 50, the snap latches 56, and the pawls 68) may be formed as separate components that are subsequently connected together or they may be integrally formed together as a unit. For example, the entire unidirectional plunger device 48 may be integrally molded from a polymer material, such as polyamide.

Among the benefits believed to be provided by the present invention is the prevention of undesirable proximal movement of the plunger 14. For example, during sterilization of the prefilled syringe body 12 (e.g., by exposure to gamma radiation), the components of a multi-part curable composition may expand, which might result in the plunger 14 being pushed proximally out of the proximal end 28 of the body 12. The plunger 14 may also move proximally during shipment (e.g., by expansion of the curable composition components when exposed to heat). Desirably, the unidirectional plunger device reduces or eliminates such unwanted proximal plunger movement without significantly impacting the movement of the plunger in the distal direction. Additionally, the unidirectional plunger device preferably provides the beneficial resistance to proximal plunger movement at various plunger positions (corresponding to various fluid levels in the channels), at least due to the continuous engagement between the device and the plunger rods as the position of the plunger within the channels varies. The inclusion of projections 66 on the device also desirably provides stability to the plunger rods 18 (as discussed above) during shipping, assembly, and injection.

The unidirectional plunger device 48 may be provided as a retrofit to an existing syringe design. For example, the base member 50 can be shaped so that it matches the shape of the proximal end 28 of one or more existing syringe designs. Similarly, the passages 64 through the base member 50 are preferably configured with the same geometry as the openings 32 in the proximal end 28 of one or more such existing syringe bodies 12. Desirably the unidirectional plunger device 48 does not interfere with the operation of the existing syringe design. In this regard, the device 48 preferably attaches to an exterior surface of the syringe body 12, such as by abutting the proximal end surface 54 of the body 12 and clipping onto a feature (e.g., lip 60) on the body 12, as discussed above. In this way, the device 48 preferably does not extend into or otherwise interfere with the interior of the syringe body 12.

Many variations of the above described embodiments are possible within the scope of the present invention. For example, the present invention is not limited to two-barrel syringes. For instance, a unidirectional plunger device in accordance with the present invention may be used in conjunction with a syringe having a single barrel or three or more barrels. In such cases, the associated components can be adjusted accordingly (e.g., by providing the appropriate number of plunger rods 18, passages 64 through the base member 50, associated pawls 68, etc.).

In other variations, the plunger rods 18 need not include teeth 72, and the ratchet mechanism may be configured to engage one or more of the plunger rods 18 so as to provide the desired unidirectional movement. For example, the free ends 70 of the pawl arms 69 may have a very sharp tip. Due to the angled, distally extending orientation of the arms 69, the tip can be arranged to slide along the smooth outer surface of the associated plunger rod 18 when the plunger 14 is moving in the distal direction, while the sharp tip can dig into and resist movement of the plunger rod 18 in the proximal direction. In such an embodiment, all or a portion of the ratchet mechanism may be constructed of a material which is substantially harder than the material of the plunger rods 18. For example, the sharp tip may be constructed of a metallic material. The above-described “toothless” embodiment may be beneficial, particularly in the case of a retrofit, as the existing plunger may not need to be modified or replaced so as to include teeth 72.

In yet other variations, the unidirectional plunger device 48 need not be removable from the syringe body 12. For example, an adhesive can be used to affix the device to the body 12, or the mechanical fixation device (e.g., snap latches) could be designed to result in permanent affixation.

Based on the design of the syringe with which the unidirectional plunger device is used, the device may extend into or be entirely located within the channels of the syringe body. For example, if the design of the syringe system is such that the cross-sectional area of the plunger rods is substantially smaller than that of the barrels, there may be sufficient space between the plunger rods and the inner periphery of the barrels to locate an embodiment of a unidirectional plunger device within the channels.

Although not shown herein, it is noted that plungers in accordance with embodiments of the present invention may include connectors located between the plunger rods and the plunger tips, as disclosed in the '451 application.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An apparatus for resisting proximal movement of a plunger of a syringe, the syringe including a body having a proximal end and a distal end, the apparatus comprising:
a base member adapted to securely attach to an exterior surface of the body of the syringe; and
a ratchet mechanism connected to the base member, the ratchet mechanism being structured and arranged to contact the plunger so as to permit distal movement of the plunger with respect to the body but resist proximal movement of the plunger with respect to the body.

2. The apparatus of claim 1, wherein the base member is shaped to securely attach to the proximal end of the body.

3. The apparatus of claim 2, wherein, when the base member is attached to the body, a shape of the base member along a plane perpendicular to a longitudinal axis of the syringe body substantially matches a shape of the syringe body along the plane.

4. The apparatus of claim 1, wherein the proximal end of the syringe body has an opening adapted to receive a portion of the plunger therethrough, and wherein the base member includes a passage therethrough adapted to receive the portion of the plunger therethrough, the passage being adapted to align with the opening at the proximal end of the syringe body when the base member is attached to the body.

5. The apparatus of claim 4, wherein the passage through the base member has substantially the same geometry as the opening at the proximal end of the syringe body.

6. The apparatus of claim 4, wherein the proximal end of the syringe body has a plurality of openings and the plunger has a plurality of plunger rods, each of the openings being adapted to receive a respective one of the plunger rods therethrough, and wherein the base member includes a plurality of passages therethrough, each of the passages being adapted to receive a respective one of the plunger rods therethrough, each of the passages being adapted to align with a respective one of the openings at the proximal end of the syringe body when the base member is attached to the body.

7. The apparatus of claim 1, further comprising a clip connected to the base member, the clip being structured and arranged to securely attach to a feature on the exterior surface of the syringe body.

8. The apparatus of claim 7, wherein the feature includes a portion of a gripping member extending away from a longitudinal axis of the syringe body.

9. The apparatus of claim 1, wherein the base member is detachable from the exterior surface of the syringe body.

10. The apparatus of claim 1, wherein the ratchet mechanism includes a pawl adapted to engage a plunger rod of the plunger when the base member is attached to the body.

11. The apparatus of claim 10, wherein the pawl is adapted to engage a plurality of teeth on the plunger rod.

12. The apparatus of claim 1, wherein the base member includes at least one projection adapted to engage a plunger rod of the plunger when the base member is attached to the body, the projection stabilizing an orientation of the plunger rod with respect to the syringe body.

13. The apparatus of claim 12, wherein the proximal end of the syringe body has an opening adapted to receive the plunger rod therethrough, and wherein the base member includes a passage therethrough adapted to receive the plunger rod therethrough, the passage being adapted to align with the opening at the proximal end of the syringe body when the base member is attached to the body, and wherein the projection extends into the passage of the base member.

14. A syringe, comprising:
a body having a proximal end, a distal end, and an exterior surface, the body including at least one barrel, each of the at least one barrel defining a fluid channel;
a plunger having at least one plunger rod, each of the at least one plunger rod being slidably receivable at least partially within a respective one of the fluid channels; and
an apparatus as recited in claim 1, the base member of the apparatus being securely attached to the exterior surface of the body.

15. The syringe of claim 14, wherein the base member is securely attached to the proximal end of the body.

16. The syringe of claim 14, wherein the base member includes at least one passage therethrough, each of the at least one passage being adapted to receive a respective one of the at least one plunger rod therethrough, and wherein the base member includes a plurality of projections extending into at least one of the passages and engaging at least one of the plunger rods to stabilize an orientation of the at least one of the plunger rods with respect to the syringe body.

17. The syringe of claim 14, wherein the body includes at least one feature on the exterior surface thereof, and wherein the apparatus includes a clip connected to the base member, the clip being securely attached to the feature.

18. The syringe of claim 14, wherein the base member is detachably attached to the exterior surface of the body.

19. The syringe of claim 14, wherein at least one of the plunger rods includes a plurality of teeth at least partially therealong, and wherein the ratchet mechanism of the apparatus includes a pawl adapted to engage the teeth.

20. A method for modifying a syringe, comprising:
securely attaching the base member of an apparatus as recited in claim 1 to an exterior surface of a body of a syringe, such that the ratchet mechanism of the apparatus contacts a plunger of the syringe so as to permit distal movement of the plunger with respect to the body but resist proximal movement of the plunger with respect to the body.
